(19) United States
(12) Patent Application Publication
(10) Pub. No.: US 2025/0327863 A1
(43) Pub. Date: Oct. 23, 2025

(54) AN ELECTRONIC MODULE OF A DRUG DELIVERY DEVICE OR OF A SUPPLEMENTAL DEVICE FOR A DRUG DELIVERY DEVICE

(71) Applicant: Sanofi, Paris (FR)

(72) Inventors: Jonathan Jason Drake, Warwick, Warwickshire (GB); Matthew Rodgers, Warwick, Warwickshire (GB)

(21) Appl. No.: 18/868,287

(22) PCT Filed: May 22, 2023
(86) PCT No.: PCT/EP2023/063543

§ 371 Date: Nov. 22, 2024

(30) Foreign Application Priority Data
May 24, 2022 (EP) 22315112.7

(51) Int. Cl. G01R 31/327; A61M 5/168

(52) U.S. Cl. CPC G01R 31/3277; A61M 5/168; A61M 2205/70

(57) ABSTRACT

Disclosed is an electronic module of a drug delivery device or of a supplemental device for a drug delivery device including: a switch configured to detect an operation performed in relation to the drug delivery device or supplemental device and to provide a respective output signal; and monitoring electronics, wherein the monitoring electronics are configured to: measure an electrical property of the switch to obtain at least one value corresponding to the electrical property; and process the at least one value to detect a fault condition of the switch.

### CROSS-REFERENCE TO RELATED APPLICATIONS

The present application is the national stage entry of International Patent Application No. PCT/EP2023/063543, filed on May 22, 2023, and claims priority to European Application No. EP 22315112.7, filed on May 24, 2022, the disclosures of which are incorporated herein by reference.

### TECHNICAL FIELD

The present disclosure relates to an electronic module of a drug delivery device or of a supplemental device for a drug delivery device, wherein the electronic module includes a switch.

### BACKGROUND

Where a switch is used in a medical device, for example to perform a detection function, the switch should perform adequately over the lifetime of the medical device such that correct operation of the switch and medical device is assured. However, switches may suffer degradation of performance over time for a variety of reasons such as wear, corrosion or erosion of the contacts of the switch, or due to contamination such as by ingress of liquids or gases. Such degradation could impede the function of the switch or reduce its reliability. This may lead to a reduction in the reliability of the medical device as a whole.

### SUMMARY

It is an object of the present disclosure to provide an electronic module of a drug delivery device or of a supplemental device for a drug delivery device, the electronic module having monitoring electronics that allow for a fault condition of a switch included by the electronic module to be detected, where the fault condition may be caused by degradation or leakage within the switch, for example. Aspects of the present disclosure may allow for a fault condition of the switch to be detected at an early stage, before the fault has developed to such an extent that it substantially affects the reliability of the switch.

According to a first aspect of the present disclosure, there is provided an electronic module of a drug delivery device or of a supplemental device for a drug delivery device, the electronic module including: a switch configured to detect an operation performed in relation to the drug delivery device or supplemental device and to provide a respective output signal; and monitoring electronics, wherein the monitoring electronics are configured to: measure an electrical property of the switch to obtain at least one value corresponding to the electrical property; and process the at least one value to detect a fault condition of the switch. Degradation or leakage in the switch may be detected at an early stage, improving the safety and/or reliability of the electronic module.

The monitoring electronics may be configured to measure the electrical property a predetermined period of time after determining that the switch has moved from either a closed state to an open state or the open state to the closed state.

The monitoring electronics may be configured to measure the electrical property a predetermined period of time after determining that the switch has moved from a closed state to an open state.

The monitoring electronics may be configured to measure the electrical property a predetermined period of time after determining that the switch has moved from an open state to a closed state.

The monitoring electronics may be configured to measure the electrical property in response to determining that the switch has moved from an open state to a closed state and while the switch remains in the closed state. The monitoring electronics may be configured to measure the electrical property while the switch has remained in the closed state after moving from the open state. This may provide a simplified and rapid means of detecting a fault condition of the switch, for example where the switch is closed during a dose dialing or dose dispensing operation of a drug delivery device.

The fault condition may include at least one of a degradation condition, indicative that electrical contacts of the switch have degraded, and a leakage condition, indicative that an electrical leakage between the electrical contacts of the switch has occurred.

Processing the at least one value to detect a fault condition of the switch may include comparing the at least one value to a threshold value and detecting the fault condition of the switch based on the comparison.

The electrical property may correspond to a voltage across the switch.

The electronic module may include a capacitor coupled across the switch. The capacitor may debounce the switch. The effect of the capacitor in delaying the time taken for a voltage across the switch to increase or decrease may be beneficial for determining a fault condition of the switch, for example by allowing a rate of voltage change to be monitored.

Measuring the electrical property of the switch may include obtaining a plurality of values corresponding to respective voltages across the switch at different respective times; and processing the at least one value to detect a fault condition of the switch may include: determining a rate of change of voltage across the switch based on the plurality of values; comparing the determined rate of change to a threshold rate of change; and detecting the fault condition of the switch based on the comparison.

The monitoring electronics may include an analogue-to-digital convertor and a processor arrangement, wherein the analogue-to-digital convertor may be configured to convert the output signal into a digital signal corresponding to the electrical property and to provide the digital signal to the processor arrangement for determining the at least one fault condition. This may provide a simple means of detecting a fault condition.

The processor arrangement may be configured to detect the fault condition by at least comparing the digital signal to a threshold.

The processor arrangement may be configured to detect the fault condition by at least determining a rate of change of the digital signal.

The monitoring electronics may be configured to generate an error signal based on the detection of a fault condition of the switch. This may allow a user to be notified of the fault condition, which may allow them to take remedial action (such as replacing the electronic module or the switch) before the fault worsens.

The electronic module may be further configured to wake up one or components of the electronic module based on the output signal.

The operation performed in relation to the drug delivery device or supplemental device may include a dose dialing operation and the electronic module may be configured to determine a dialled dose based on the output signal. Such an operation would require a high reliability of the switch to ensure an accurate dialled dose is determined, and therefore aspects of the present disclosure may be beneficial in such a scenario in that they may detect a fault condition before the reliability of the switch is substantially reduced.

The operation performed in relation to the drug delivery device or supplemental device may include a dose dispensing operation and the electronic module may be configured to determine a dispensed dose based on the output signal. Such an operation would require a high reliability of the switch to ensure an accurate dispensed dose is determined, and therefore aspects of the present disclosure may be beneficial in such a scenario in that they may detect a fault condition before the reliability of the switch is substantially reduced.

The processor arrangement may be configured to detect the fault condition by comparing the at least one measured value to a trend or profile of values, and the fault condition may be detected based at least in part on whether the at least one measured value corresponds to or deviates from the trend or profile.

The trend or profile of values may include a trend or profile of historical values that have been previously measured for the switch.

According to a second aspect of the present disclosure, there is provided a drug delivery device or a supplemental device attachable to a drug delivery device, including any electronic module described herein.

According to a third aspect of the present disclosure, there is provided a method including: measuring, by monitoring electronics of an electronic module of a drug delivery device or of a supplemental device for a drug delivery device, an electrical property of a switch of the electronic module to obtain at least one value corresponding to the electrical property, wherein the switch is configured to detect an operation performed in relation to the drug delivery device or supplemental device and to provide a respective output signal; and processing, by the monitoring electronics, the at least one value to detect a fault condition of the switch.

Measuring the electrical property may be performed a predetermined period of time after determining that the switch has moved from a closed state to an open state.

Measuring the electrical property may be performed a predetermined period of time after determining that the switch has moved from an open state to a closed state.

Measuring the electrical property may be performed in response to determining that the switch has moved from an open state to a closed state that the switch has remained in the closed state after moving from the open state.

Detecting the fault condition may include comparing the at least one measured value to a trend or profile of values, and the fault condition may be detected based at least in part on whether the at least one measured value corresponds to or deviates from the trend or profile.

The trend or profile of values may include a trend or profile of historical values that have been previously measured for the switch.

### BRIEF DESCRIPTION OF THE FIGURES

Exemplary embodiments will now be described with reference to the accompanying drawings, of which:

FIG. 1 shows an example of a drug delivery device in accordance with aspects of the present invention;

FIG. 2 shows an example of a drug delivery add-on device attached to a drug delivery device in accordance with aspects of the present invention;

FIG. 3 is a schematic diagram of various electronic components with an electronic module according to aspects of the present invention;

FIG. 4A is a schematic circuit diagram of part of a sensing arrangement including a switch in accordance with aspects of the present invention;

FIG. 4B is a schematic circuit diagram showing the same part of a sensing arrangement as FIG. 4A, but also illustrating the effect degradation of the switch has on the circuit;

FIG. 4C is a schematic circuit diagram showing the same part of a sensing arrangement as FIG. 4A, but also illustrating the effect electrical leakage in the switch has on the circuit;

FIG. 4D is a schematic circuit diagram showing the same part of a sensing arrangement as FIG. 4A, but also illustrating the effects both degradation of the switch and electrical leakage in the switch have on the circuit;

FIG. 5 is a schematic circuit diagram of electronic components for an electronic module in accordance with aspects of the present invention;

FIG. 6 is a flow chart illustrating a method to be performed by the electronic module in accordance with aspects of the present invention;

FIG. 7 is a graph showing the change in voltage across the switch of FIG. 5 over time, as the switch is moved from an open state to a closed state;

FIG. 8 is a graph showing the change in voltage across the switch of FIG. 5 over time, as the switch is moved from a closed state to an open state.

### DETAILED DESCRIPTION

Aspects of the present disclosure may provide a means of monitoring switch performance in a medical device such that a fault condition of the switch (such as due to degradation of the switch or electrical leakage within the switch) can be detected and communicated for interpretation.

In the following, embodiments of the present disclosure will be described with reference to injection devices, particularly an injection device in the form of an injection pen. The present disclosure is however not limited to such application and may equally well be deployed with drug delivery devices other than an injection device, and with shapes other than a pen.

In the following, some embodiments will also be described with reference to an insulin injection device. The present disclosure is however not limited to such application and may equally well be deployed with injection devices that are configured to eject other medicaments or drug delivery devices in general.

Embodiments are provided in relation to injection devices, in particular to variable dose injection devices, which record and/or track measurement data on doses delivered thereby, or to which a supplemental device may be attached to record and/or track measurement data on doses delivered thereby. These data may include the size of the selected dose and/or the size of the actually delivered dose, the time and date of administration, the duration of the administration and the like.

Certain embodiments in this document are illustrated with respect to the injection device disclosed in EP 2 890435 where an injection button and grip (dose setting member, dose setter or dosage knob) are combined. The injection button may be actuated by a user to initiate and/or perform a dose delivery operation of the drug delivery device. The grip or knob may be used by a user to initiate and/or perform a dose setting operation. These injection devices may be of the dial extension type, i.e. their length increases during dose setting. However, the general principles of the present disclosure are not limited to that kinematical behaviour. Certain other embodiments may be conceived for application to Sanofi's SoloSTAR® injection device where there are separate injection button and grip components/dose setting members. Thus, there may be two separate user interface members, one for the dose setting operation and one for the dose delivery operation.

“Distal” is used herein to specify directions, ends or surfaces which are arranged or are to be arranged to face or point towards a dispensing end of the drug delivery device or components thereof and/or point away from, are to be arranged to face away from or face away from the proximal end. On the other hand, “proximal” is used to specify directions, ends or surfaces which are arranged or are to be arranged to face away from or point away from the dispensing end and/or from the distal end of the drug delivery device or components thereof. The distal end may be the end closest to the dispensing and/or furthest away from the proximal end and the proximal end may be the end furthest away from the dispensing end. A proximal surface may face away from the distal end and/or towards the proximal end. A distal surface may face towards the distal end and/or away from the proximal end. The dispensing end may be the needle end where a needle unit is or is to be mounted to the device, for example.

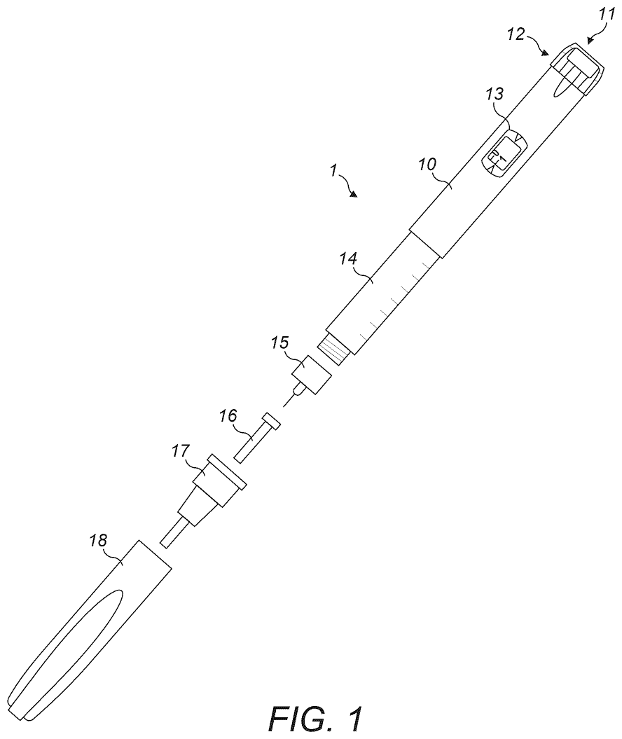

FIG. 1 is an exploded view of a medicament delivery device or drug delivery device including an electronic module 11 according to aspects of the present disclosure. In this example, the medicament delivery device is an injection device 1, e.g. a pen-type injector, such an injection pen disclosed in EP 2 890435.

The injection device 1 of FIG. 1 is an injection pen that includes a housing 10 and contains a container 14, e.g. an insulin container, or a receptacle for such a container 14. The container 14 may contain a drug. A needle 15 can be affixed to the container 14 or the receptacle. The container 14 may be a cartridge and the receptacle may be a cartridge holder. The needle 15 may be protected by at least one of an inner needle cap 16, an outer needle cap 17, or another cap 18. An insulin dose to be ejected from injection device 1 can be set, programmed, or ‘dialled in’ by turning an injection button or dial grip (dosage knob) 12, and a currently programmed or set dose is then displayed via dosage window 13, for instance in multiples of units. The indicia displayed in the window 13 may be provided on a number sleeve or dial sleeve partially visible through the window 13. For example, where the injection device 1 is configured to administer human insulin, the dosage may be displayed in so-called International Units (IU), wherein one IU is the biological equivalent of about 45.5 micrograms of pure crystalline insulin ( 1/22 mg). Other units may be employed in injection devices for delivering analogue insulin or other medicaments. It should be noted that the selected dose may equally well be displayed differently than as shown in the dosage window 13 in FIG. 1.

The dosage window 13 may be in the form of an aperture in the housing 10, which permits a user to view a limited portion of a dial sleeve assembly that is configured to move when the button or dial grip 12 is turned, to provide a visual indication of a currently set dose. The button or dial grip 12 may be rotated on a helical path with respect to the housing 10 when setting a dose.

The injection device 1 may be configured so that turning the button or dial grip 12 causes a mechanical click sound to provide acoustic feedback to a user. In this embodiment, the button or dial grip 12 also acts as an injection button. When needle 15 is stuck into a skin portion of a patient, and then the button or dial grip 12 is pushed in an axial direction, the insulin dose displayed in display window 13 will be ejected from injection device 1. When the needle 15 of injection device 1 remains for a certain time in the skin portion after the button or dial grip 12 is pushed, the dose is injected into the patient's body. Ejection of the insulin dose may also cause a mechanical click sound, which may be different from the sounds produced when rotating the button or dial grip 12 during dialing of the dose.

In this example, during delivery of the insulin dose, the button or dial grip 12 is returned to its initial position in an axial movement, without rotation, while the dial sleeve assembly is rotated to return to its initial position, e.g. to display a dose of zero units. FIG. 1 shows the injection device 1 in this 0U dialled condition. As noted already, the disclosure is not restricted to insulin but should encompass all drugs in the drug container 14, especially liquid drugs or drug formulations.

Injection device 1 may be used for several injection processes until either the insulin container 14 is empty or the expiration date of the medicament in the injection device 1 (e.g. 28 days after the first use) is reached. In the case of a resuable injection device 1, it is possible to replace the insulin container.

Before using injection device 1 for the first time, it may be necessary to perform a so-called “prime shot” to remove air from insulin container 14 and needle 15, for instance by selecting two units of insulin and pressing button or dial grip 12 while holding injection device 1 with the needle 15 upwards. For simplicity of presentation, it will be assumed in the following that the ejected amounts substantially correspond to the injected doses, so that, for instance the amount of medicament ejected from the injection device 1 is equal to the dose received by the user. Nevertheless, differences (e.g. losses) between the ejected amounts and the injected doses may need to be taken into account.

As explained above, the button or dial grip 12 also functions as an injection button so that the same component is used for dialling/setting the dose and dispensing/delivering the dose. As an alternative (not shown), a separate injection button may be used which is axially displaceable, at least a limited distance, relative to a dial grip 12 to effect or trigger dose dispensing.

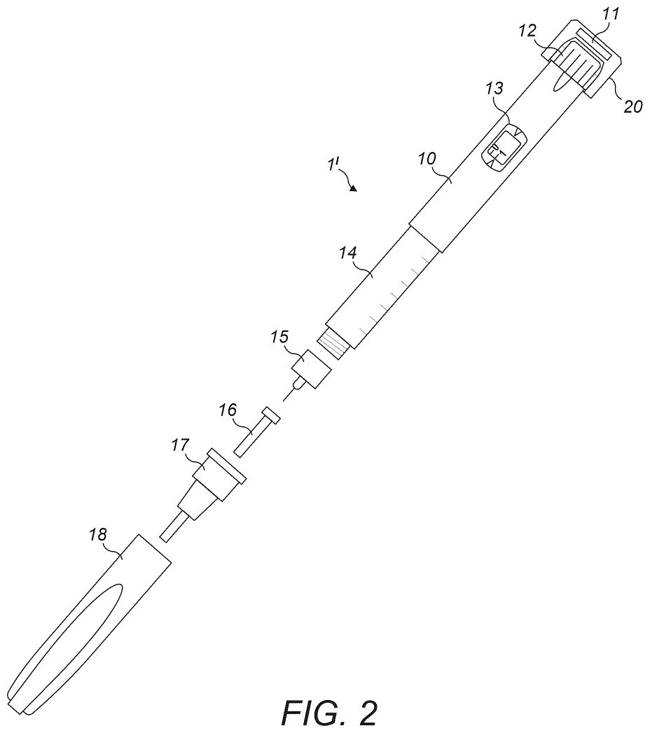

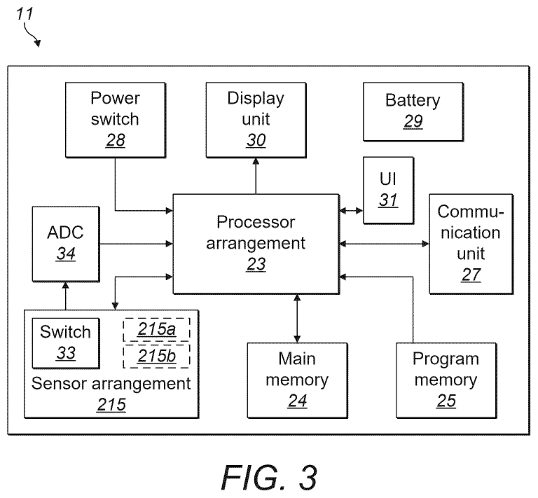

FIG. 1 shows an electronic module 11 contained within the injection device 1, in particular in the button or dial grip 12 of the injection device 1. However, in some examples the electronic module 11 may be located within a different part of the injection device 1, or in a supplemental device 20 attachable to the injection device as later described in relation to FIG. 2. In other examples, the electronic module 11 may be split between the injection device 1 and the supplemental device 20 such that some components of the electronic module 11 are included in the injection device 1 while the remaining components of the electronic module 11 are included in the supplemental device 20. The electronic module 11 is described later throughout this application, for example in relation to FIG. 3.

In some examples, the button or dial grip 12 may include one or more formations to facilitate attachment of a supplemental device 20 (otherwise known as add-on device), for example a data collection device. FIG. 2 shows an injection device 1′ similar to the injection device 1 of FIG. 1, however a supplemental device 20 (shown in cross-section) is attached to the injection device. In this example, the supplemental device 20 takes the form of a button module and is coupled to the button or dial grip 12 of the injection device 1′. The supplemental device 20 may be coupled to the button or dial grip 12 of the injection device 1′ such that a user may apply a force to the button or dial grip 12 via the supplemental device 20 in order to eject a dose of medicament. In other words, a user may push the supplemental device 20 in an axial direction towards the injection device 1 such that the axial force of the push is transferred from the supplemental device 20 to the button or dial grip 12 to cause dispensing of medicament as previously described in relation to FIG. 1.

While FIG. 2 shows the supplemental device 20 taking the form of a button module coupled to the button or dial grip 12 of the injection device 1′, in other examples the supplemental device 20 may take a different form and/or be attached to a different part of the injection device 1′ such as the housing 10 or an injection button separate from the dial grip 12. The supplemental device 20 may be configured to releasably attach to the injection device 1′, or permanently attach to the injection device 1′. As shown in FIG. 2, the supplemental device may contain the electronic module 11.

In the following, an electronic module 11 according to the present disclosure will be described with respect to exemplary embodiments and with reference to FIGS. 1 to 8.

As depicted in FIG. 3, the electronic module 11 may include a processor arrangement 23 including one or more processors, such as a microprocessor, a Digital Signal Processor (DSP), Application Specific Integrated Circuit (ASIC), Field Programmable Gate Array (FPGA) or the like, and one or more memory units 24, 25 such as program memory 25 and main memory 24, which can store software for execution by the processor arrangement 23 and may store data acquired, processed or generated by the electronic module 11 or another device in communication with the electronic module 11.

The electronic module 11 may further include a communication unit or output 27. The communication unit 27 may include a wireless communications interface for communicating with another device such as a mobile phone via a wireless network such as Wi-Fi™ or Bluetooth®, and/or an interface for a wired communications link, such as a socket for receiving a Universal Series Bus (USB), mini-USB or micro-USB connector. Other forms of wired and/or wireless communications interface may be used.

The electronic module 11 may further include a display unit 30, for example a LCD (Liquid Crystal Display), one or more LEDs, and/or an electronic paper display. The electronic module 11 may include a user interface (UI) 31 for receiving a user input to the electronic module 11, wherein the user interface 31 may include one or more buttons and/or touch input devices, for example.

The electronic module 11 further includes a battery 29 for supplying power to one or more electrical components of the electronic module 11, although other means of supplying power may be used instead of or in addition to a battery 29, for example a receiver coil for receiving wireless energy, or a capacitor. The electronic module 11 may include a power switch 28 for variably connecting one or more electrical components of the electronic module 11 to the battery 29, however in some examples a power switch 28 is not present.

The electronic module 11 includes a sensor arrangement 215 including a switch 33. The switch 33 may be an electromechanical switch such as a momentary switch, a detect switch, a push button switch, a toggle switch, a rocker switch, a rotary switch, or a slider switch, although other forms of electromechanical switch may be used.

In some examples the switch 33 is the power switch 28 described previously, but in other examples the switch 33 is a separate switch from the power switch 28, or else the electronic module 11 includes a switch 33 but no power switch 28.

The switch 33 includes at least a pair of electrical contacts. The switch 33 is movable between a closed state in which, in normal operation, an electrical current may pass through the switch 33 between the electrical contacts, and an open state in which, in normal operation, an electrical current cannot pass through the switch 33 between the electrical contacts (or only a negligible current may pass). The switch 33 may be movable from the closed state to the open state, and/or vice versa. The switch 33 is configured to provide an output signal corresponding to whether it is in the open state or closed state. The output signal may be provided to another component of the electronic module 11, such as the processor arrangement 23, to be processed.

The switch 33 may be used to detect an operation (such as a mechanical operation/process) performed in relation to the supplemental device 20 and/or injection device 1. For example, the switch 33 may be configured to detect a dose dialing operation and/or a dose dispensing operation. The switch 33 may be configured to detect a dose dialing operation and/or a dose dispensing operation by being arranged within the injection device 1 and/or supplemental device 20 such that the switch is actuated (an hence moved from the open state to closed state or vice versa) by movement (e.g. linear or rotational) of a movable component of the injection device 1 during the dose dialing operation and/or a dose dispensing operation. The movable component may be a dial sleeve or dial grip 12, however other types of movable component may be used instead. The output signal from the switch 33 may be processed by the processor arrangement 23 to detect the operation. For example, the output signal may be processed to determine whether a dose dialing operation and/or a dose dispensing operation has occurred, or to determine a value for a dialed dose and/or dispensed dose, for example.

In some examples the detected operation may be a user-initiated operation to ‘wake up’ one or more components of the electronic module 11, or a device coupled to the electronic module. Waking up the components may include causing said components to move from a no-power state or a relatively low-power state to a relatively high-power state. As examples, the user may actuate the switch 33 to wake up one or more additional sensors of the sensor arrangement, or to move the processor arrangement 23 from a relatively low power state (in which operations of the processor arrangement 23 are limited) to a relatively high power state (in which operations of the processor arrangement 23 are less limited). Actuating the switch 33 may cause an output signal to be sent from the switch 33 to the processor arrangement 23, causing the processor arrangement 23 to wake up one or more components of the electronic module 11 or a device coupled to the electronic module 11.

In some examples, the detected operation may include a user applying a force to the supplemental device 20, for example to cause a medicament dose to be expelled by the injection device 1 (such as previously described in relation to FIG. 2), to cause a dose to be dialed, or to perform some other action such as waking up the electronics of the supplemental device 20. The switch 33 will be located so as to detect such an operation. For example, the switch 33 may be located on a lower surface of the supplemental device 20 such that it may contact the button or dial grip 12 of the injection device 1 and be actuated when a user applies a force to the button or dial grip 12 via the supplemental device 20. However, in other examples the switch 33 may be located elsewhere, for example at an upper surface of the supplemental device 20 or a location within the supplemental device 20 such that the force is relayed through the supplemental device 20 and detected by the switch 33.

The sensor arrangement 215 may include one or more additional sensors or sensing components such as optical sensors, magnetic sensors, acoustic sensors, capacitive sensors or vibration sensors, although other types of sensors may be used. For example, the sensor arrangement may include an LED 215a and a photo detector 215b together forming an optical sensor. The one or more additional sensors may be configured to determine a dose dialed into the injection device 1 and/or a dose dispensed by the injection device 1, for example where these operations are not performed by the switch 33. The one or more additional sensors may be configured to determine the dialed dose and/or dispensed dose by detecting movement (e.g. linear or rotational) of a movable component of the injection device 1 before, during and/or after a dose dialing operation and/or a dose dispensing operation. The movable component may be a dial sleeve or dial grip 12, however other types of movable component may be used instead. As an example, the sensor arrangement 215 may include an optical encoder arranged to output a signal corresponding to an amount of movement (linear or rotational) of a dial sleeve, wherein the signal may be processed for example by the processor arrangement 23 to determine a value for a dialed dose or dispensed dose. In other examples, a capacitive sensor may detect the movement.

One or more components of the sensor arrangement 215 may be controlled by the processor arrangement 23, or by one or more devices of the sensor arrangement 215.

The electronic module 11 further includes monitoring electronics, for monitoring a condition of the switch 33. The monitoring electronics are configured to measure an electrical property of the switch 33 to obtain at least one value corresponding to the electrical property, and to process the at least one value to detect a fault condition of the switch 33.

The electrical property may be a voltage across the switch 33 and the at least one value may be at least one value corresponding to the voltage across the switch. In other examples, the electrical property may be a current through the switch 33 and the at least one value may be at least one value corresponding to the current through the switch 33. By monitoring a condition of the switch 33 it is meant that the monitoring electronics are configured to detect a fault condition of the switch 33, wherein the fault condition may include one or more of degradation condition of the switch 33 (i.e. that the electrical contacts of the switch 33 have degraded, for example by at least a predetermined amount) or electrical leakage condition of the switch 33 (i.e. that electrical leakage between the electrical contacts of the switch 33 is occurring). Degradation of the switch 33 and electrical leakage are discussed later in the application.

The monitoring electronics may include the processor arrangement 23 and any suitable components for measuring the electrical property, for example an analog-to-digital converter (ADC) 34 as shown in FIG. 3. The ADC 34 is configured to receive as an input from the switch 33 an analogue electrical signal which may correspond to a voltage at one side of the switch 33. The analogue electrical signal may correspond to the voltage across the switch 33. The electrical signal may be the output signal of the switch 33 described previously. The ADC 34 is configured to convert the input electrical signal into a digital signal corresponding to the voltage across the switch 33, and output said digital signal to the processor arrangement 23 for further processing. Operation of the ADC 34 is described elsewhere in this application. While it is described herein that the monitoring means may include an ADC 34, it should be understood that in some examples an ADC 34 is not present, and the monitoring means includes one or more different components for measuring an electrical property of the switch 33, such as a suitable arrangement of logic components.

Any of the components of the electronic module 11 shown in FIG. 3 may be soldered on a PCB containing wiring for electronically coupling the components. Some of the components such as the processor arrangement 23, main memory 24, program memory 25, communication unit 27 and ADC 34 may be included by a SoC (System on Chip) or microcontroller. As discussed previously, the components of the electronic module 11 may be included in an injection device 1 such as in the button or dial grip 12 of an injection device 1, or they may be included in a supplemental device 20 configured to be attached to an injection device 1′. However, in some examples the components of the electronic module 11 may be distributed between an injection device 1 and a supplemental device 20. For example, the processor arrangement 23 may be included in a supplemental device 20 while the sensor arrangement 215 is included in the injection device 1. Other distributions of the components of the electronic module 11 may be envisaged.

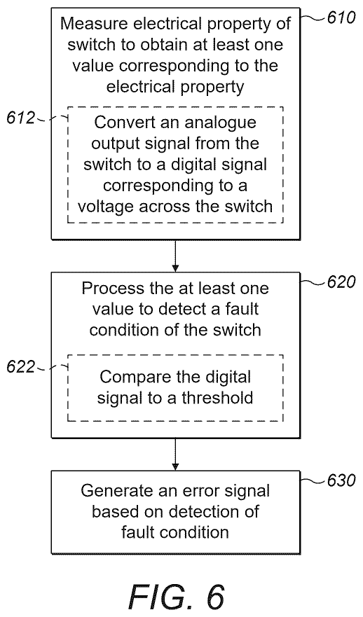

A firmware stored in the program memory 25 may configure the processor arrangement 23 to cause one or more of the method steps disclosed herein to be performed (such as the method discussed in relation to FIG. 6), and/or control the operation of one or more other components of the electronic module 11 such as the ADC 34 or sensor arrangement 215.

The switch 33 is configured to provide an output signal which is dependent on whether the switch 33 is in the closed state or the open state. The output signal may be provided to the processor arrangement 23 for further processing. The output signal may be processed by the processor arrangement 23 to determine whether to wake up one or more components of the electronic module 11, to determine a dose of medicament dialed into or dispensed from the injection device 1, or determine whether a dose dispense operation or dose dialing operation has taken place, for example.

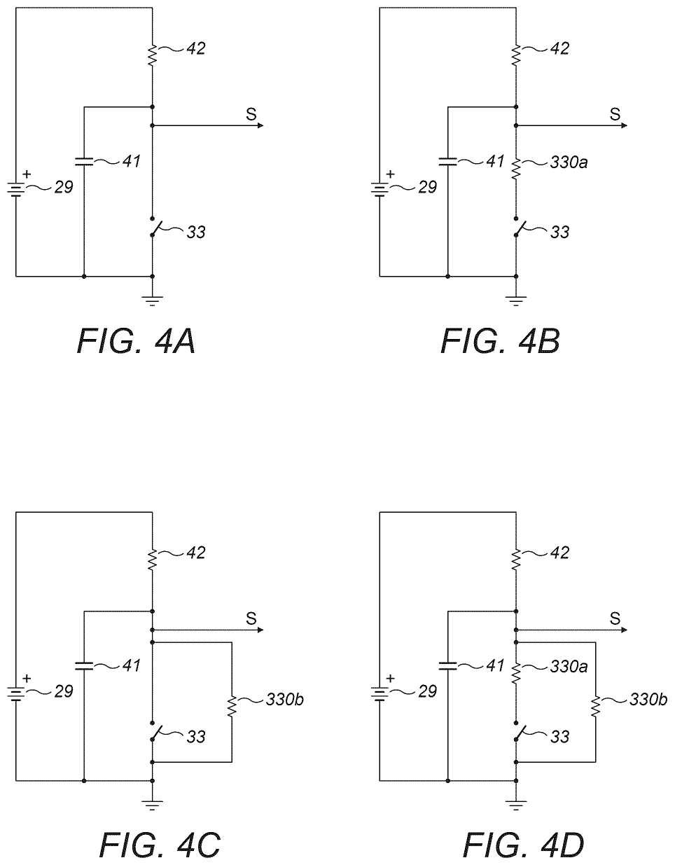

In some examples, the output signal of the switch 33 may be high (i.e. include a non-negligible voltage or current) when the switch 33 is in the open state, and may be low (i.e. include a zero or negligible voltage or current) when the switch 33 is in the closed state. To provide a suitable output signal for use by the processor arrangement 23 and/or the ADC 34, the switch 33 may be included in the sensor arrangement 215 as part of a potential divider (voltage divider), as illustrated in the schematic circuit diagram of FIG. 4A.

FIG. 4A shows the switch 33 arranged in a series circuit with a pull-up resistor 42 and an electrical power supply, such as a battery 29. The output signal S is provided via an electrical connection connected to a center tap of the potential divider formed by the resistor 42 and the switch 33.

If the pull-up resistor 42 has a fixed resistance R1 and the switch 33 has a resistance R2, then the voltage Vout of the output signal S provided at the center tap of the potential divider (i.e. the voltage across the switch 33) may be determined using the following equation:

$${V}_{out} = {V}_{in} ( \frac{{R}_{2}}{{R}_{1} + {R}_{2}} )$$

where Vᵢₙ is the input voltage provided by the battery 29 to the potential divider (i.e. to the resistor 42).

For an ideal switch 33 (i.e. with no faults/degradation/leakage) in an open state as shown in FIG. 4A, the resistance R2 of the switch 33 will be infinite (or effectively infinite). Therefore assuming the pull-up resistor 42 has a resistance R1 substantially less than R2, the voltage Vout of the output signal S will be approximately equal to Vin. The voltage Vout of the output signal S will therefore be high (i.e. a non-negligible voltage).

For the ideal switch 33 (i.e. with no faults/degradation/leakage) in a closed state, the resistance R2 of the switch 33 will be lower than the resistance R2 of the switch 33 in the open position. The resistance R2 of the switch 33 in the closed state may be close to zero, but will not be zero due to inherent resistances in the components of the switch 33 such as the electrical contacts (even without degradation). Approximating the resistance R2 of the switch 33 in the closed position to be zero and assuming the resistance R1 of the pull-up resistor 42 is non-zero and non-negligible, the voltage Vout of the output signal S will be approximately equal to zero. The voltage Vout of the output signal S will therefore be low (i.e. a zero or negligible voltage).

The output signal S can therefore be processed to determine whether the switch 33 is in an open state or a closed state, for example by providing the output signal S to a processor arrangement 23, which may detect whether the output signal S is high or low, and therefore whether the switch 33 is open or closed.

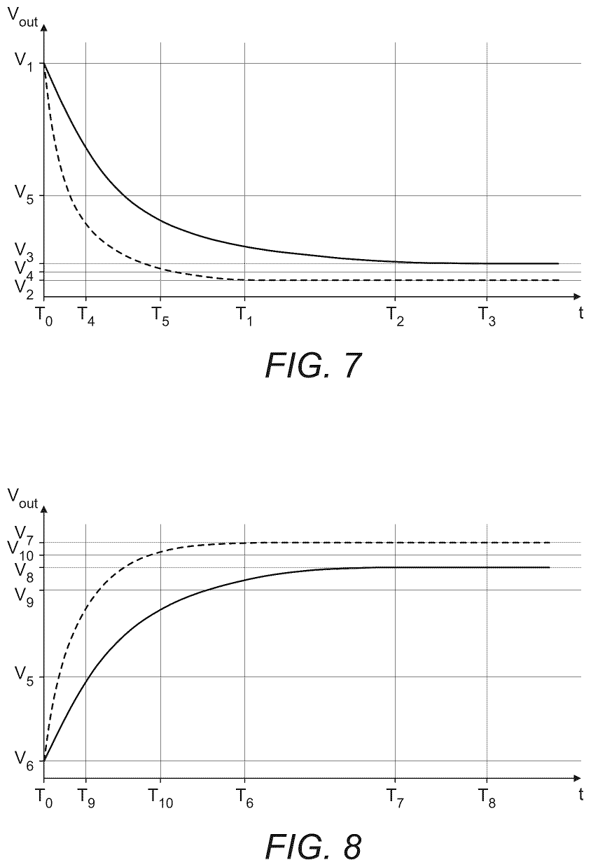

The potential divider of the sensor arrangement 215 may also have a capacitor 41 connected in parallel with the switch 33, as shown in FIG. 4A. The capacitor 41 may be used for debouncing the output signal S of the switch 33. The presence of the capacitor 41 will increase the time taken for Vout to change to a new value when the switch 33 is moved from the closed state to open state or vice versa, as later described in relation to FIGS. 7 and 8.

It should be noted that if the polarity of the battery 29 shown in FIG. 4A was reversed or the positions of the switch 33 and resistor 42 reversed then the previous references to ‘high’ and ‘low’ voltages and signals would be reversed. Therefore it should be understood that the terms ‘high’ and ‘low’ generally correspond to the magnitude of the voltages and may not necessarily be indicative of polarity.

The output signal S may be provided to the processor arrangement 23 for processing as discussed previously. Based on a property of the output signal (e.g. whether the output signal is high or low), the processor arrangement 23 may determine whether the switch 33 is in an open state or a closed state, and consequently may use this information to wake up one or more electrical components of the electronic module 11 or determine a dose of medicament dialed and/or ejected, for example.

FIG. 4B is a schematic circuit diagram showing generally the same circuit as FIG. 4B, however the circuit of FIG. 4B illustrates a scenario in which the switch 33 of FIG. 4A has degraded from its normal or ideal state. The electrical contacts of the switch 33 may have degraded (deteriorated) due to being worn, corroded, eroded, dirty, or otherwise in a poor state due to one or more other factors. The deterioration of the contacts may cause an increase in the resistance of the switch 33. This increased resistance is illustrated in FIG. 4B as an effective (virtual) resistor 330a connected in series with the switch 33. The effect of the increased resistance of the switch 33 is that, when the switch 33 is in the closed state, the voltage Vout of the output signal S will be greater than voltage Vout of the output signal S described in relation to FIG. 4A (i.e. the ‘ideal’ situation without degradation of the switch). Moving the switch 33 from the open state to the closed state will still cause Vout of the output signal S to decrease, however the decrease will be smaller than in the scenario illustrated in FIG. 4A. The voltage Vout of the output signal S when the switch 33 is in the closed state will be non-zero and likely non-negligible. As the switch 33 further deteriorates over time, the resistance of the closed switch 33 may further increase, resulting in a higher value for Vout when the switch 33 is in the closed state.

The increased resistance of the switch 33 will also lead to an increase in the time taken for Vout to go low after the switch 33 is closed, because the debounce capacitor 41 must be discharged through a higher resistance provided by the degraded switch 33.

In some extreme cases, the effective resistance of the closed switch 33 could be increased to such a large extent through degradation that operation of the switch 33 from the open state to the closed state or vice versa is not reliably detected by the processor arrangement 23. Aspects of the present disclosure may seek to avoid such a scenario, for example by detecting deterioration of the switch 33 early, before it is able to affect the reliability of the switch and therefore reliability of the electronic module 11, as a whole.

FIG. 4C is a schematic circuit diagram similar to that shown in FIG. 4A, however the circuit diagram illustrates a scenario in which electrical leakage is occurring between the electrical contacts of the switch 33, effectively shorting the switch 33. The leakage may be the result of the ingress of an electrically conducting fluid into the switch 33, or another part of the electronic module 11, for example. The leakage acts an impedance in parallel with the electrical contacts of the switch 33, as illustrated by the effective resistor 330b in FIG. 3C. The effect of the leakage is to decrease the voltage Vout of the output signal S when the switch 33 is in the open state, when compared to the scenario of FIG. 4A. The leakage will also result in the voltage Vout of the output signal S increasing at a slower rate after the switch 33 has moved from the closed state to open state In some extreme cases, excess leakage could inhibit detection of the switch 33 moving from the open state to the closed state or vice versa. The leakage may also unnecessarily drain power from the battery 29 when the switch 33 is in the open state. In some cases the leakage could be a temporary condition. For example, if the leakage is caused by fluid ingress, the fluid may over time evaporate or drain away, reducing the effects of the leakage over time.

In some cases, it is possible for the switch 33 to simultaneously suffer from degradation of the electrical contacts and from leakage. This is illustrated in FIG. 4D, which is a schematic circuit diagram similar to FIG. 4A, but showing an effective resistor 330c in series with the switch 33 and an effective resistor 330d in parallel with the switch 33. The effective resistor 330c represents degradation of the electrical contacts of the switch 33 while the effective resistor 330d represents an electrical leakage across the switch 33.

The deterioration of the contacts and the leakage may present as random perturbations rather than ideal, constant, phenomena. For example, leakage due to ingress of fluid may vary depending upon the geometric factors of the electrical contacts of the switch 33 in relation to the liquid and other parameters which may be highly variable such as, but not limited to, the fluid composition and temperature. Where aqueous fluids are present and interacting with the contacts of the switch 33, an electrochemical potential could be set up which may induce an additional voltage that pushes the signal voltage Vout higher or lower than expected from a purely resistive interaction.

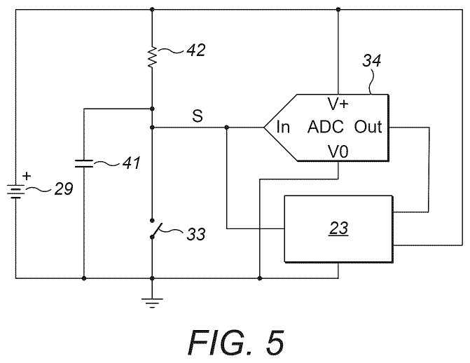

FIG. 5 is a schematic circuit diagram showing example components of an electronic module 11 according to an embodiment of the present disclosure, wherein the electronic module 11 may be able to detect a fault condition of the switch 33 caused by at least one of degradation or leakage as previously described in relation to FIGS. 4A-D.

The circuit shown in FIG. 5 is similar to the circuit shown in FIG. 4A, as indicated by the presence of the battery 29, pull-up resistor 42, capacitor 41 and switch 33. The circuit of FIG. 5 further includes monitoring electronics including an ADC 34 and a processor arrangement 23, which may be the ADC 34 and processor arrangement 23 described previously in relation to FIG. 3 and elsewhere in this disclosure. The circuit of the electronic module 11 may include further components such as one or more memory units 24, 25, however such further components are not included in FIG. 5 for simplicity.

FIG. 5 shows the battery 29 providing supply power to the ADC 34 and the processor arrangement 23, in addition to the potential divider formed by the resistor 42 and switch 33. However, in other examples one or more of the ADC 34, the processor arrangement 23 and the potential divider may use a different power supply to the others. In some examples, the ADC 34 uses the power supply voltage provided by the battery 29 to the potential divider as its reference voltage. This may compensate for fluctuations in power supply voltage, improving the accuracy with which a fault condition of the switch 33 may be detected by the electronic module 11.

As illustrated in FIG. 5, the analogue output signal S from the switch 33 is provided as an input to both the ADC 34 and the processor arrangement 23. This may be via the same electrical connection from the central tap of the potential divider formed by the resistor 42 and the switch 33. However, in other examples, a signal from the switch 33 to the processor arrangement 23 may be relayed in a different manner.

The ADC 34 receives as an input the output signal S from the switch 33 and converts the analogue output signal S to a digital signal, which is output by the ADC 34 to the processor arrangement 23. In particular, the ADC 34 converts an analogue input voltage or current provided by the output signal S to a digital number representing the magnitude of the voltage or current, with this digital number provided to the processor arrangement 23 in the digital signal. As an example, the greater the input voltage provided to the ADC 34, the greater the digital number output by the ADC 34. The processor arrangement 23 is able to determine a voltage Vout of the output signal S from the switch 33 (i.e. the voltage across the switch 33) based on the digital signal, for example by using a lookup table stored in a memory unit 24, 25. The lookup table may include a plurality of digital numbers and the corresponding values for Vout they represent.

As previously discussed in relation to FIGS. 4A-4D, the value of Vout can be influenced by the degradation of the electrical contacts of the switch 33 and/or electrical leakage across the electrical contacts of the switch 33. The processor arrangement 23 may therefore be able to process the digital signal to detect a fault condition of the switch 33. A fault condition of the switch 33 may include a degradation condition, in which the electrical contacts of the switch 33 have degraded (deteriorated) and therefore have increased in resistance, a leakage condition, in which an electrical leakage between the contacts of the switch 33 has occurred, or a combination of the degradation condition and leakage condition. Example methods of processing the digital signal are discussed in relation to FIGS. 6-8.

The ADC 34 may continuously sample the output signal S and provide a digital signal output to the processor arrangement 23. However, in some examples the ADC 34 samples the output signal S on demand and at discrete intervals, for example in response to a command signal being received from the processor arrangement 23. This may increase the energy efficiency of the monitoring electronics.

FIG. 6 is a flow chart illustrating a method of determining a fault condition of a switch 33 according to aspects of the present disclosure. The method may be performed by an electronic module 11 as described elsewhere in this application, for example an electronic module 11 including the circuit illustrated in FIG. 5. The steps may be performed by the monitoring electronics, that is the processor arrangement 23 and/or ADC 34.

In step 610, an electrical property of the switch 33 is measured to obtain at least one value representing the electrical property. The electrical property has been described in this disclosure as a voltage Vout across the switch 33. The at least one value may therefore be at least one value representing a voltage Vout across the switch 33. However, in other examples the electrical property may be a current passing through the switch 33, or a different electrical property that is influenced by degradation or leakage of the switch contacts, for example.

If the monitoring electronics included an ADC 34 then measurement of the electrical property may be carried out using the ADC 34. In this case, step 610 may include an optional step 612, in which an analogue output signal S from the switch 33 is converted to a digital signal corresponding to a voltage across the switch 33. The conversion may be performed by the ADC 34, in some examples in response to an instruction from the processor arrangement 23. The at least one value obtained by the measurement may be at least one digital number output by the ADC 34 in its digital signal.

In some examples, the step of measuring the electrical property of the switch 33 may include obtaining a plurality of values corresponding to respective voltages across the switch 33 at different respective times, as described later in relation to FIGS. 7 and 8. This may allow a rate of change of the voltage across the switch 33 to be determined.

In some examples, the monitoring electronics are configured to measure the electrical property a predetermined period of time after determining that the switch 33 has moved from either a closed state to an open state or the open state to the closed state. This may allow the output signal (i.e. the voltage of the output signal) time to settle, which may improve the accuracy of the measurement. Allowing the output signal time to settle may be of particular importance when a debounce capacitor 41 is coupled across the switch 33, since the presence of the capacitor 41 will cause the output signal to take longer to settle. The electronic module 11 may determine that the switch 33 has moved from either a closed state to an open state or the open state to the closed state by monitoring the output signal S of the switch 33. For example, it might be determined by the electronic module 11 (e.g. the processor arrangement 23 of the electronic module 11) that the switch 33 has moved from a closed state to an open state in response to the processor arrangement 23 detecting that the voltage of the output signal has increased. There are a variety of manners in which this may be achieved, for example by the processor arrangement 23 determining that the voltage of the output signal S has increased above a threshold voltage (such as 0V), that the voltage has increased by at least a threshold amount or a threshold percentage, or that the voltage has increased by at least a threshold amount or a threshold percentage within a certain period of time. Similarly, it might be determined by the electronic module 11 (e.g. the processor arrangement 23 of the electronic module 11) that the switch 33 has moved from an open state to a closed state in response to the processor arrangement 23 detecting that the voltage of the output signal has decreased. There are a variety of manners in which this may be achieved, for example by the processor arrangement 23 determining that the voltage of the output signal S has decreased below a threshold voltage (such as 0.9V), that the voltage has decreased by at least a threshold amount or a threshold percentage, or that the voltage has decreased by at least a threshold amount or a threshold percentage within a certain period of time. The monitoring electronics may be configured to measure the electrical property responsive to a positive determination by the monitoring electronics that the switch 33 has moved from either the closed state to the open state or the open state to the closed state. The measurement may take place a predetermined period of time after the positive determination.

In step 620, the at least one value is processed, for example by the processor arrangement 23, to detect a fault condition of the switch 33. Processing the at least one value may include determining that the at least one value deviates from an expected value or range of values. For example, processing the at least one value may include comparing the at least one value to at least one threshold value, and detecting the fault condition based on the comparison. In some examples the at least one threshold value may be predetermined. In other examples, the at least one threshold value may be determined based on previous measurements of the electrical property of the switch 33, that is previous values representing historical measurements of the electrical property of the switch 33.

If optional step 612 has taken place, then step 620 may include an optional step 622, in which the digital signal output by the ADC 34 is compared to a threshold. More particularly, a digital number included in the digital signal may be compared by the processor arrangement 23 to a threshold value or range of threshold values, for example stored in a lookup table. The processor arrangement 23 may detect a fault condition of the switch 33 based on the comparison, for example in response to determining at least one of the digital number being above a threshold value, being below a threshold value, or being within a range of threshold values.

If a plurality of values corresponding to respective voltages across the switch 33 at different respective times were obtained in step 610, then step 620 may include determining a rate of change of voltage across the switch 33 based on the plurality of values. The determined rate of change may be compared to a threshold rate of change, and the fault condition of the switch 33 may be detected based on the comparison, for example if the determined rate is below a threshold rate.

Various examples of processing the at least one value to detect a fault condition are described later in relation to FIG. 7 and FIG. 8.

In an optional step 630, an error signal may be generated based on the detection of the fault condition. The error signal may be generated by the processor arrangement 23, and the error signal may be generated in response to the detection of the fault condition. In some examples, the error signal may be used to generate an alert to a user, to indicate that the switch 33 of the electronic module 11 has degraded or is in a leakage state. The alert may therefore indicate to the user that the device containing the electronic module 11 (such as the injection device 1 or supplemental device 20) should perhaps be replaced or repaired. The alert may include at least one of an audio, visual, or haptic alert, for example. The alert may be output by the electronic module 11 under the control of the processor arrangement 23, for example as a visual alert via the display unit 30, an audio alert via an audio output device such as a speaker, or a haptic alert via a haptic output device. In some examples, the processor arrangement 23 may output the error signal to an external device such as a mobile phone or computer, for example via the communication unit 27. The error signal may cause an alert to be output by said external device. As an example, the processor arrangement 23 may output the error signal as an encoded wireless message via the communication unit 27 using a Bluetooth protocol. The encoded Bluetooth message may be received by the external device, which may decode and process the message and issue a corresponding alert.

FIG. 7 is a graph illustrating the change in voltage across the switch 33 as a function of time when in the circuit shown in FIG. 5, demonstrating the effect of degradation of the electrical contacts of the switch 33. Vout is the voltage of the signal output S (i.e. the voltage across the switch 33).

The dotted line represents the voltage across a switch 33 having no degradation of the electrical contacts of the switch 33. This may be a newly-manufactured switch 33, for example, and shall be referred to as an ‘ideal’ switch 33. Such a switch 33 may correspond to the scenario previously described in relation to FIG. 4A. The solid line in FIG. 7 represents the voltage across the same switch 33 after the electrical contacts of the switch 33 have undergone degradation, thereby increasing the inherent resistance of the switch 33 when closed. Such a switch 33 shall be referred to as the ‘degraded’ switch 33, and may correspond to the scenario previously described in relation to FIG. 4B.

Time T0 represents an initial time when the switch 33 is in an open state. It is assumed for simplicity in this example that there is no electrical leakage across the electrical contacts of the ideal switch 33 or the degraded switch 33. As such, the initial voltage across both the ideal switch 33 and the degraded switch 33 is the same, shown in FIG. 7 as V1. If Vout is to be provided as an input to the processor arrangement 23 for determination whether the switch 33 is in a closed or open state, then V1 may be approximately 1V, for example.

Once the ideal switch 33 is closed, the voltage across the ideal switch 33 decreases until it reaches a new steady voltage V2 at time T1. V2 is non-zero due to the inherent resistance of the switch 33 (not caused by degradation), nevertheless V2 is close to zero due to no additional resistance being provided by degradation. The decrease in voltage Vout between T0 and T1 is gradual rather than instantaneous, due to the presence of the debounce capacitor 41 coupled across the switch 33. The voltage across the ideal switch 33 will remain substantially constant at V2 for as long as the ideal switch 33 remains closed after time T1.

For comparison, once the degraded switch 33 is closed at time T0, the voltage across the degraded switch 33 also decreases in a gradual manner until it reaches a new lower voltage, however this new lower voltage V3 is higher than the voltage V2 of the ideal switch 33 due to the degradation of the electrical contacts of the degraded switch 33 increasing the resistance of the degraded switch 33 compared to the ideal switch 33. Furthermore, it will take the degraded switch 33 until time T2 to reach the steady voltage V3, wherein T2 occurs a period of time after T1.

In order to detect a fault condition of switch 33 (e.g. degraded switch 33), an electronic module 11 according to the present disclosure may measure the voltage V3 across the switch 33 a predetermined period of time after the switch 33 has moved from the open state to the closed state, i.e. at a time T3 after T0. Time T3 is chosen to be a period of time after the switch 33 has been moved from the open state to the closed state at time T0, and in some cases may be chosen so as to provide sufficient time for the voltage across the switch 33 to settle to a new constant level V3. The period of time may be predetermined, for example 0.1 seconds. It may be determined by the processor arrangement 23 that the switch 33 has moved from the open state to the closed state by detecting that the voltage across the switch 33 has fallen by a predetermined amount, a predetermined percentage, or below a predetermined threshold, with voltage V3 across the switch 33 then being measured a period of time after that detection. Various manners of detecting that the switch 33 has moved from the open state to the closed state were previously discussed in relation to FIG. 6.

Once a value for the voltage V3 across the switch 33 has been measured, it may then be processed to detect a fault condition of the switch 33, as discussed in relation to step 620 of FIG. 6. For example, the value for the measured voltage V3 may be compared to a predetermined threshold voltage V4, wherein V4 is chosen to be greater than V2 (to allow for minor deviations from the ideal voltage V2). A fault condition of the switch 33 may be detected if it is determined from the comparison that the measured voltage V3 is greater than the threshold value V4. In this case, the fault condition is a condition signifying that the electrical contacts of the switch 33 have degraded, for example by more than an acceptable amount. In some examples, V4 may be around 0.1V. It may therefore be detected that the switch 33 is in a degradation condition if it is determined that the value of V3 is greater than 0.1V at time T3 after T0. In other examples, V4 may be around 0V.

In some examples, the measured voltage V3 is compared to more than one threshold value, for example threshold V4 and threshold V5, where V5 corresponds to a voltage which is higher than V4 but less than the initial open switch voltage V1. Detecting a fault condition of the switch 33 may include comparing V3 to threshold V4 and threshold V5, by determining whether V4>V3>V5 is satisfied. If V4>V3>V5 is satisfied, then the fault condition is a condition signifying that the electrical contacts of the switch 33 have degraded. In some examples V4 and V5 may represent 0V and 0.1V respectively, while in other examples they may represent 0.1V and 0.2V respectively, although other values may be envisaged.

It can be seen in FIG. 7 that the voltage across the degraded switch 33 decreases at a smaller rate than the voltage across an ideal (non-degraded) switch 33 when moved from the open state to the closed state, as illustrated by the shallower gradient of solid line compared to the dashed line. This is due to the debounce capacitor 41 taking longer to discharge through the degraded switch 33 having a relatively higher resistance compared to the ideal switch 33 having the relatively low resistance. Aspects of the present disclosure may take advantage of this phenomenon to detect a fault condition or state of the switch 33. Therefore in some examples, measuring an electrical property of the switch 33 may include obtaining more than one value of voltage across the switch 33 at different respective times, and determining a rate of change of the voltage based on the obtained voltage values. For example, measurements of voltage across the degraded switch 33 may be taken at times T4 and T5 after the switch 33 has moved from the open state to the closed state. A rate of change of voltage may be determined based on the difference between the two voltage measurements and the difference between the times T4 and T5. Detecting a fault condition of the switch 33 may then include comparing the determined rate with a threshold rate. The threshold rate may have been chosen such that if the determined rate is less than the threshold rate, a fault condition of the switch 33 is considered to be detected, wherein the fault condition is a degradation condition signifying that the electrical contacts of the switch 33 have degraded.

FIG. 8 is a graph illustrating the change in voltage across the switch 33 as a function of time when in the circuit shown in FIG. 5, demonstrating the effect of electrical leakage across the electrical contacts of the switch 33. Vout is the voltage of the signal output S (i.e. the voltage across the switch 33).

The dotted line represents the voltage across a switch 33 having no electrical leakage across the electrical contacts of the switch 33. This may be a newly-manufactured switch 33, for example, and shall be referred to as an ‘ideal’ switch. Such a switch 33 may correspond to the scenario previously described in relation to FIG. 4A. The solid line represents the voltage across the same switch 33 if electrical leakage between the electrical contacts of the switch 33 is present, thereby decreasing the inherent resistance of the switch 33 when in an open state. Such a switch 33 shall be referred to as the ‘leaking’ switch, and may correspond to the scenario previously described in relation to FIG. 4C.

At time T0 shown in FIG. 8, both the ideal switch 33 and the leaking switch 33 are moved from their respective closed states to their respective open states. When in their closed states, the voltage across both the ideal switch 33 and leaking switch 33 is similar, shown as voltage V6 at time T0. Once the ideal switch 33 is opened, the voltage across the ideal switch 33 increases until it reaches a steady state voltage of V7 at time T6. In a similar manner, once the leaking switch 33 is opened, the voltage across the leaking switch 33 increases until it reaches a steady state voltage of V8 at time T7. It can be seen that V8 is less than V7 on account of the leakage between the electrical contacts of the leaking switch 33 reducing the effective resistance of the leaking switch 33 compared to the ideal switch 33. The leaking switch 33 also takes longer than the ideal switch 33 to reach its steady state voltage, as shown in FIG. 8 by T7 being later than T6, and the rate of increase in voltage being smaller for the leaking switch 33 than the ideal switch 33, due to the leakage causing the debounce capacitor 41 to take longer to recharge.

In order to detect a fault condition of switch 33 (e.g. leaking switch 33), an electronic module 11 according to the present disclosure may measure the voltage V8 across the switch 33 a predetermined period of time after the switch 33 has moved from the closed state to the open state, i.e. at a time T8 after T0. Time T8 is chosen to be a period of time after the switch 33 has been moved from the closed state to the open state at time T0, and in some cases may be chosen to provide sufficient time for the voltage across the switch 33 to settle to a new, higher constant level V8. The period of time may be predetermined, for example 0.1 seconds. It may be determined by the processor arrangement 23 that the switch 33 has moved from the closed state to the open state by detecting that the voltage across the switch 33 has increased by a predetermined amount, a predetermined percentage, or above a predetermined threshold, with voltage V8 across the switch 33 then being measured the predetermined period of time of time after that detection. Various manners of detecting that the switch 33 has moved from the closed state to the open state were previously discussed in relation to FIG. 6.

Once a value for the voltage V8 across the switch 33 has been measured, it may then be processed to detect a fault condition of the switch 33, as discussed in relation to step 620 of FIG. 6. For example, the value for the measured voltage V8 may be compared to a predetermined threshold voltage V10, wherein V10 is chosen to be less than V7 (to allow for minor deviations from the ideal voltage V7). A fault condition of the switch 33 may be detected if it is determined from the comparison that the measured voltage V8 is less than the threshold value V10. In this case, the fault condition is a leakage condition signifying that electrical leakage between the electrical contacts of the switch 33 is occurring, for example by more than an acceptable amount. In some examples, V10 may be around 0.9V. It may therefore be detected that the switch 33 is in a leakage condition if it is determined that the value of V8 is less than 0.9V at time T8 after T0. In other examples, V10 may be around 1V.

In some examples, the measured voltage V8 is compared to more than one threshold value, for example threshold V10 and threshold V9, where V9 corresponds to a voltage which is less than V10 but greater than the initial open switch voltage V6. Detecting a fault condition of the switch 33 may include comparing V8 to threshold V9 and threshold V10, by determining whether V10>V8>V9 is satisfied. If V10>V8>V9 is satisfied, then the fault condition is a leakage condition signifying that electrical leakage is occurring between the electrical contacts of the switch 33. In some examples V9 and V10 may represent 0.8V and 0.9V respectively, while in other examples they may represent 0.9V and 1V respectively, although other values may be envisaged.

It can be seen in FIG. 8 that the voltage across the leaking switch 33 increases at a smaller rate than the voltage across an ideal (non-leaking) switch 33 when moved from the open state to the closed state, as illustrated by the shallower gradient of the solid line compared to the dashed line. This is due to the debounce capacitor 41 taking longer to recharge when current is leaking through the leaking switch 33. Aspects of the present disclosure may take advantage of this phenomenon to detect a fault condition or state of the switch 33. Therefore in some examples, measuring an electrical property of the switch 33 may include obtaining more than one value of voltage across the switch 33 at different respective times, and determining a rate of change of the voltage based on the obtained voltage values. For example, measurements of voltage across the degraded switch 33 may be taken at times T9 and T10 after the switch 33 has moved from the closed state to the open state. A rate of change of voltage may be determined based on the difference between the two voltage measurements and the difference between the times T9 and T10. Detecting a fault condition of the switch 33 may then include comparing the determined rate with a threshold rate. The threshold rate may have been chosen such that if the determined rate is less than the threshold rate, a fault condition of the switch 33 is considered to be detected, wherein the fault condition is a leakage condition signifying that the electrical leaking is occurring between the contacts of the switch 33.

While it has been discussed in relation to FIGS. 7 and 8 that processing the at least one measured value corresponding to a voltage may include comparing the at least one measured value to one or more thresholds or ranges, it should be understood that the at least one measured value may be processed in a different manner for detecting a fault condition of the switch 33. For example, the at least one measured value may be compared to a trend or profile of values, and the fault condition may be detected based at least in part on whether the at least one measured value corresponds to or deviates from the trend or profile. For example, the trend or profile of values may include a trend or profile of historical values that have been previously measured for the switch 33, and the fault condition may be detected based at least in part on whether the at least one measured value deviates from the trend or profile, for example by certain amount. In other examples, the trend or profile of values may include a trend or profile of predicted values for the switch 33, and the fault condition may be detected based at least in part on whether the at least one measured value deviates from the trend or profile, for example by certain amount. Other methods and/or statistical processes might be used to process and interpret the at least value to detect a fault condition.

While embodiments described herein have discussed various processing steps (such as step 620) as being performed by the processor arrangement 23, it should be noted that in other examples the processing may be carried out by a different component of the electronic module 11, or indeed by an external device such as a mobile device. In this case, the electronic module 11 may be configured to transmit data including the at least one value obtained by the monitoring electronics in step 610 to the external device, for example via the communication unit 27, so that the external device may process the at least one value to detect the fault condition of the switch. The electronic module 11 may store the at least one value in a memory 24, 25 prior to transmission to the external device.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des (B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not include a full-length antibody polypeptide, but that still includes at least a portion of a full-length antibody polypeptide that is capable of binding to an antigen. Antibody fragments can include a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1: 2014(E). As described in ISO 11608-1: 2014(E), needle-based injection systems may be broadly distinguished into multi-dose container systems and single-dose (with partial or full evacuation) container systems. The container may be a replaceable container or an integrated non-replaceable container.

As further described in ISO 11608-1: 2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1: 2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1: 2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

1.-20. (canceled)

21. An electronic module of a drug delivery device or of a supplemental device for a drug delivery device, the electronic module comprising:
a switch configured to detect an operation performed in relation to the drug delivery device or supplemental device and to provide a respective output signal; and
monitoring electronics,
wherein the monitoring electronics are configured to:
measure an electrical property of the switch to obtain at least one value corresponding to the electrical property; and
process the at least one value to detect a fault condition of the switch.

22. An electronic module according to claim 21, wherein the monitoring electronics are configured to measure the electrical property a predetermined period of time after determining that the switch has moved from a closed state to an open state.

23. An electronic module according to claim 21, wherein the monitoring electronics are configured to measure the electrical property a predetermined period of time after determining that the switch has moved from an open state to a closed state.

24. An electric module according to claim 21, wherein the monitoring electronics are configured to measure the electrical property in response to determining that the switch has moved from an open state to a closed state and while the switch remains in the closed state.

25. An electronic module according to claim 21, further comprising a debounce capacitor coupled across the switch.

26. An electronic module according to claim 21, wherein the fault condition comprises at least one of a degradation condition, indicative that electrical contacts of the switch have degraded, and a leakage condition, indicative that an electrical leakage between the electrical contacts of the switch has occurred.

27. An electronic module according to claim 21, wherein processing the at least one value to detect a fault condition of the switch comprises comparing the at least one value to a threshold value and detecting the fault condition of the switch based on the comparison.

28. An electronic module according to claim 21, wherein the electrical property corresponds to a voltage across the switch.

29. An electronic module according to claim 28,
wherein measuring the electrical property of the switch comprises obtaining a plurality of values corresponding to respective voltages across the switch at different respective times; and
wherein processing the at least one value to detect a fault condition of the switch comprises:
determining a rate of change of voltage across the switch based on the plurality of values;
comparing the determined rate of change to a threshold rate of change; and
detecting the fault condition of the switch based on the comparison.

30. An electronic module according to claim 21, wherein the monitoring electronics comprise an analogue-to-digital convertor and a processor arrangement,
wherein the analogue-to-digital convertor is configured to convert the output signal into a digital signal corresponding to the electrical property and to provide the digital signal to the processor arrangement for determining the at least one fault condition.

31. An electronic module according to claim 30, wherein the processor arrangement is configured to detect the fault condition by at least comparing the digital signal to a threshold.

32. An electronic module according to claim 30, wherein the processor arrangement is configured to detect the fault condition by at least determining a rate of change of the digital signal.

33. An electronic module according to claim 21, wherein the monitoring electronics are configured to generate an error signal based on the detection of a fault condition of the switch.

34. An electronic module according to claim 21, wherein the electronic module is further configured to wake up one or components of the electronic module based on the output signal.

35. An electronic module according to claim 21, wherein:
the operation performed in relation to the drug delivery device or supplemental device comprises a dose dialing operation, and the electronic module is configured to determine a dialled dose based on the output signal.

36. An electronic module according to claim 21, wherein:
the operation performed in relation to the drug delivery device or supplemental device comprises a dose dispensing operation, and the electronic module is configured to determine a dispensed dose based on the output signal.

37. An electronic module according to claim 21, wherein the processor arrangement is configured to detect the fault condition by comparing the at least one measured value to a trend or profile of values, and the fault condition is detected based at least in part on whether the at least one measured value corresponds to or deviates from the trend or profile.

38. An electronic module according to claim 37, wherein the trend or profile of values comprises a trend or profile of historical values that have been previously measured for the switch.

39. A drug delivery device or a supplemental device attachable to a drug delivery device, comprising an electronic module, comprising
a switch configured to detect an operation performed in relation to the drug delivery device or supplemental device and to provide a respective output signal; and
monitoring electronics,
wherein the monitoring electronics are configured to:
measure an electrical property of the switch to obtain at least one value corresponding to the electrical property; and
process the at least one value to detect a fault condition of the switch.

40. A method, comprising:
measuring, by monitoring electronics of an electronic module of a drug delivery device or of a supplemental device for a drug delivery device, an electrical property of a switch of the electronic module to obtain at least one value corresponding to the electrical property, wherein the switch is configured to detect an operation performed in relation to the drug delivery device or supplemental device and to provide a respective output signal; and
processing, by the monitoring electronics, the at least one value to detect a fault condition of the switch.
